(19) United States
(12) Patent Application Publication
(10) Pub. No.: US 2011/0212042 A1
(43) Pub. Date: Sep. 1, 2011

(54) SKIN CARE COMPOSITION HAVING DESIRABLE BULK COLOR

(76) Inventors: Prithwiraj Maitra, Randolph, NJ (US); Anna Prilutsky, Princeton, NJ (US)

(21) Appl. No.: 13/036,094

(22) Filed: Feb. 28, 2011

Related U.S. Application Data
(60) Provisional application No. 61/309,060, filed on Mar. 1, 2010.

(51) Int. Cl. A61K 8/29; A61Q 1/02

(52) U.S. Cl. 424/63

(57) ABSTRACT

The invention relates to a skin care composition, such as a color cosmetic, comprising an active ingredient that imparts an undesirable color to the composition, at least one inorganic pigment that comprises at least 60 weight percent titanium dioxide, at least one lake pigment, and at least one interference pigment.

### CROSS REFERENCE TO RELATED APPLICATIONS

This application claims priority of the benefits of the filing of U.S. Provisional Application Ser. No. 61/309,060 filed Mar. 1, 2010. The complete disclosure of the aforementioned related patent application is hereby incorporated herein by reference for all purposes.

### FIELD OF THE INVENTION

The present invention relates to skin care compositions, such as color cosmetics, comprising an active ingredient having an undesirable color, at least one inorganic pigment that comprises at least 60 weight percent titanium dioxide, at least one lake pigment, and at least one interference pigment. Despite the color of the active ingredient, the overall skin care composition has a consumer acceptable shade.

### BACKGROUND OF THE INVENTION

It is often desirable to include additives, such as active ingredients, to skin care compositions to provide additional benefits to the skin. For example, WO 2009/045720 and US 2007/0060862 disclose topical compositions comprising galvanic particulates and a variety of benefits provided thereby. WO 2009/045720 discloses that galvanic particulates may increase soft tissue volume by increasing collagen or elastin in the skin or lips.

Similarly, U.S. Pat. No. 6,410,062 discloses methods of treating inflammatory disorders using topical compositions containing extracts of feverfew.

However, certain active ingredients, such as galvanic particulates or feverfew extract, may impart undesirable colors to skin care compositions, which may be negatively affect their consumer appeal. Galvanic powder may give skin care compositions a dark, metallic, or grey color. Feverfew extract may impart a yellow or brown color.

Applicants have now discovered that the presence of active ingredients having an undesirable color in skin care compositions may be masked using certain ingredients and methods. In particular, applicants have discovered that combination of such an active ingredient with at least one inorganic pigment that comprises at least 60 weight percent titanium dioxide, at least one lake pigment, and at least one interference pigment provides a cosmetic composition having a desirable, pleasant color in bulk, and a consumer acceptable shade when applied to the skin.

### SUMMARY OF THE INVENTION

The invention relates to a skin care composition comprising:
a) an active ingredient having an undesirable color;
b) about 0.05% to 4 weight percent of at least one inorganic pigment, wherein said inorganic pigment comprises at least 60 weight percent titanium dioxide;
c) about 0.02 to 1.5 weight percent of at least one lake pigment; and
d) about 0.05 to 4.5 weight percent of at least one interference pigment.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference. Unless otherwise indicated, a percentage refers to a percentage by weight (i.e., % (W/W)).

As used herein, “cosmetically-acceptable” means suitable for use in topical contact with tissues (e.g., the skin) without undue toxicity, incompatibility, instability, irritation, allergic response, or the like. This term is not intended to limit the composition it describes as for use solely as a cosmetic (e.g., the composition may be used as a pharmaceutical).

As used herein, “safe and effective amount” means an amount sufficient to provide a desired benefit at a desired level, but low enough to avoid serious side effects.

As used herein, the term “treating” or “treatment” means alleviation or elimination of symptoms, cure, prevention, or inhibition of a human condition or disease, specifically of the skin.

The skin care composition may be any cosmetically-acceptable formulation. It may take any one of a wide variety of forms that include but are not limited to lotions, creams, gels, sticks, sprays, shaving creams, ointments, cleansing liquid washes and solid bars, shampoos, pastes, powders, mousses, shaving creams, wipes, patches, nail lacquers, wound dressings and adhesives, hydrogels, films, serum, moisturizers, and color cosmetics.

In one embodiment, the skin care composition is a color cosmetic. As used herein, “color cosmetic” means a composition for application to the hair, nails and/or skin, especially the face, which contains at least about 0.01% and up to about 50% of pigment. Color cosmetics include, but are not limited to, foundations, concealers, primers, blush, mascara, eyeshadow, eyeliner, lipstick, nail polish and tinted moisturizers. The present invention is particularly suited for use with primers.

As used herein, “foundation” means a liquid, solid, or semi-solid cosmetic composition for imparting color to the skin, especially the face. It may be in the form of a lotion, cream, gel, serum, compact, stick, or paste.

As used herein, “concealer” means a liquid, paste, or semi-solid cosmetic composition for imparting color to the skin, containing a relatively high level of pigments having opacity, such as titanium dioxide, typically used prior to applying foundation, for example for concealing age or acne spots or scars.

As used herein, “primer” means a liquid, paste, or semi-solid cosmetic composition for application directly to the skin underneath foundations and/or concealers. Primers ease the application of foundation (or other skin care composition) onto the skin, even out skin tone, and increase the longevity of skin care compositions applied over the primer. Primers also may be used to smooth fine lines, such as around the mouth. A lip primer used underneath lipstick can maintain lip color and prevent feathering of the lipstick. Foundation primer used around the eye area can decrease creasing of eyeshadow. Use of a foundation primer may also decrease the amount of foundation required to achieve the same effect. Primers typically comprise waxes, polymers, and silicones.

The skin care composition comprises at least one ingredient, such as an active ingredient, having an undesirable color.

In one embodiment, the active ingredient comprises a plant extract or other natural ingredient. Examples of plant extracts include, but are not limited to, soy, glycine soja, oatmeal, what, aloe vera, cranberry, witch-hazel, alnus, arnica, artemisia capillaris, asiasarum root, birch, calendula, chamomile, cnidium, comfrey, fennel, galla rhois, hawthorn, houttuynia, hypericum, jujube, kiwi, licorice, magnolia, olive, peppermint, philodendron, salvia, sasa albo-marginata, natural isoflavonoids, soy isoflavones, and natural essential oils.

In another embodiment, the active ingredient comprises feverfew extract. As used herein, “feverfew extract” is a blend of compounds isolated from a plant from the Chrysanthemum or Tanacetum genus (hereinafter referred to as feverfew). Examples of feverfew include, but are not limited to, Chrysanthemum parthenium, Tanacetum parthenium, or Matricania parthenium, as well as those listed in CRC Ethnobotany Desk Reference 1998, ed. Timothy Johnson, p 198-199, 823-824, 516-517 (CRC Press, Boca Raton, Fla., USA 1998) and the The Plant Names Project (1999), International Plant Names Index, published on the Internet; http://www.ipni.org [accessed Jan. 11, 2001].

Such compounds may be isolated from a part(s) of the plant (e.g., the arial part of the plant such as the stem, flower, and leaves) by physically removing a piece of such plant, such as grinding a leaf on the plant. Such compounds may also be isolated from the plant by using extraction procedures well known in the art (e.g., the use of organic solvents such as C1-C8 alcohols, C1-C8 alkyl polyols, C1-C8 alkyl ketones, C1-C8 alkyl ethers, acetic acid C1-C8 alkyl esters, and chloroform, and/or inorganic solvents such as water, inorganic acids such as hydrochloric acid, and inorganic bases such as sodium hydroxide). In one embodiment, the feverfew extract contains only hydrophilic compounds (e.g., isolated by using a hydrophilic solvent, such as water or ethanol). In one embodiment, the feverfew extract contains only hydrophobic compounds (e.g. isolated by using a hydrophobic solvent, such as chloroform). In one embodiment, the feverfew extract contains both hydrophilic and hydrophobic compounds.

In one embodiment, the feverfew extract is substantially free of alpha-unsaturated gamma-lactones. The term “substantially free of alpha-unsaturated gamma-lactones,” refers to a feverfew extract having a weight content of the alpha-unsaturated gamma-lactones of less than about 0.2% by weight. These alpha-unsaturated gamma-lactones include, but are not limited to, parthenolide, 3-β-hydroxy-parthenolide, costunolide, 3-β-constunolide, artemorin, 8-α-hydroxy-estafiatin, chysanthemolide, magnoliolide, tanaparthin, tanaparthin-1α,4α-epoxide, tanaparthin-1β,4β-epoxide, chrysanthemonin, and other sesquiterpenes. Preferably, the feverfew extract has a weight content of alpha-unsaturated gamma-lactones below about 0.02% by weight.

Alpha-unsaturated gamma-lactones, including parthenolide, are present in feverfew. Methods for the manufacture of feverfew extracts that are substantially free of parthenolide and other alpha-unsaturated gamma-lactones are disclosed in PCT Patent Application No. WO 00/74695.

The amount of the feverfew extract present in the composition will depend on the type of extract used. In one embodiment, the composition comprises a safe and effective amount of said feverfew extract. The extract typically will be present in the composition in an amount from about 0.001% to about 20% by weight

In one embodiment, the composition is substantially free of parthenolide. What is meant by “substantially free of parthenolide” is that the composition comprises, by weight, less than 0.1%, preferably below 0.01%, more preferably below 0.001% or does not comprise any parthenolide. In one embodiment, the composition does not comprise parthenolide.

In another embodiment, the active ingredient comprises a vitamin. Examples of vitamins include Vitamin E, Vitamin A, Vitamin C, Vitamin B, and salts or derivatives thereof such as ascorbic acid di-glucoside and vitamin E acetate or palmitate.

In another embodiment, the active ingredient comprises a,b,d,g-tocopherol. For example, the active ingredient may be COVI-OX T 70C commercially available from Cognis.

In another embodiment, the active ingredient comprises a copper peptide. As used herein, “copper peptide” is a peptide complexed with a copper ion. Examples of such copper peptides are set forth in U.S. Pat. Nos. 4,665,054, 4,760,051, 4,810,693, 4,877,770, 5,135,913, 5,348,943, 5,382,431, and 5,550,183. In one embodiment, the peptide has from 3 to 10 amino acids. In one embodiment, the peptide is of the Formula I:

$$[ \begin{matrix}R 1 \\ R 2\end{matrix} > A 1 \text{-} A 2 \text{-} His \text{-} A 3 \text{-} A 4 \text{-} R 3 ] n : copper ( II ) \qquad Formula 1$$

wherein A1 is Gly or absent; A2 is Gly, Lys, Ala, Ser, or Val; A3 is Lys or Gly; A4 is Trp, (Gly)n-Trp where n is from 1 to 4, Pro-Val-Phe-Val, Val-Phe-Val, or absent; each R1 and R2, independently, is H, C₁₋₁₂ alkyl, C₇₋₁₀ phenylalkyl, or C(═O)E1, where E1 is C₁₋₂₀ alkyl, C₃₋₂₀ alkenyl, C₃₋₂₀ alkynyl, phenyl, 3,4-dihydroxyphenylalkyl, naphthyl, or C₇₋₁₀ phenylalkyl; provided that when either R1 or R2 is C(═O)E1, the other must be H; R3 is OH, NH₂, C₁₋₁₂ alkoxy, C₇₋₁₀ phenylalkoxy, C₁₁₋₂₀ naphthylalkoxy, C₁₋₁₂ alkylamino, C₇₋₁₀ phenylalkylamino, or C₁₁₋₂₀ naphthylalkylamino; and n is 1 or 2. Copper (II) may be bound to one or more counter anions. Examples of additional counter anions include, but are not limited to, halides such as chloride, acetates, phosphonates, and sulfates, e.g., copper diacetate.

In one embodiment, A1 is absent. In one embodiment, A2 is Gly, Lys, or Ala. In one embodiment, A3 is Lys or Gly. In one embodiment, A4 is absent. In one embodiment, R1 and R2 are both H. In one embodiment, R3 is OH, NH₂, or C₁₋₁₂ alkoxy.

In one embodiment, the peptide is [H₂-Gly-His-Lys-OH]n:copper(II), [H₂-Gly-His-Lys-NH₂]n:copper(II) (Copper Tripeptide-1), [H₂-Ala-His-Lys-OH]n:copper(II), or [H₂-Ala-His-Lys-NH₂]n:copper(II).

The symbol A1, A2, or the like used herein (e.g., in Formula I) stands for the residue of an alpha-amino acid. Such symbols represent the general structure, —NH—CH(X)—CO— or ═N—CH(X)—CO— when it is at the N-terminus or —NH—CH(X)—CO— when it is not at the N-terminus, where X denotes the side chain (or identifying group) of the alpha-amino acid, e.g., X is —CH(CH₃)₂ for Val. Note that the N-terminus is at the left and the C-terminus at the right in accordance with the conventional representation of a polypeptide chain. R1 and R2 are both bound to the free nitrogen atom N-terminal amino acid (e.g., A1 or A2) and the R3 is bound to the free carboxy group of the C-terminal amino acid (e.g., A3 or A4). Further, where the amino acid residue is optically active, it is the L-form configuration that is intended unless the D-form is expressly designated. An alkyl group, if not specified, contains 1-12 carbon atoms.

The amount of the copper peptide present in the composition will depend on the copper peptide used and the intended use of the composition. In one embodiment, the composition comprises a safe and effective amount of the copper peptide. The copper peptide is typically present in an amount from about 0.001% to about 20% by weight, in particular in an amount from about 0.01% to about 1% by weight.

Methods for synthesizing copper peptides are well known, for example as described in in U.S. Pat. Nos. 4,810,693 and 5,550,183.

In one embodiment, the active ingredient has a dark, metallic, or grey color.

In one embodiment, the skin care composition comprises galvanic particulates. Each galvanic particulate comprises a first conductive material and a second conductive material, wherein both the first conductive material and the second conductive material are exposed on the surface of the galvanic particulate. In one embodiment, the galvanic particulates comprise the first conductive material partially coated with the second conductive material.

Preferably, the skin care composition comprises up to about 10 weight percent galvanic particulates, for example up to about 5 weight percent galvanic particulates or up to about 1 weight percent galvanic particulates.

In one embodiment, the galvanic particulates are produced by a coating method wherein the weight percentage of the second conductive material is from about 0.001% to about 20%, by weight, of the total weight of the particulate, such as from about 0.01% to about 10%, by weight, of the total weight of galvanic particulate. In one embodiment, the coating thickness of the second conductive material may vary from single atom up to hundreds of microns. In yet another embodiment, the surface of the galvanic particulate comprises from about 0.001 percent to about 99.99 percent such as from about 0.1 to about 99.9 percent of the second conductive material.

In one embodiment, the galvanic particulates are produced by a non-coating method (e.g., by sintering, printing or mechanical processing the first and the second conductive materials together to form the galvanic particulate) wherein the second conductive material comprises from about 0.1% to about 99.9%, by weight, of the total weight of the particulate, such as from about 10% to about 90%, of the total weight of the particulate.

In one embodiment, the galvanic particulates are fine enough that they can be suspended in semi-solid compositions during storage. In a further embodiment, they are in flattened and/or elongated shapes. The advantages of flattened and elongated shapes of the galvanic particulates include a lower apparent density and, therefore, a better floating/suspending capability in the skin care composition, as well as better coverage over the skin, leading to a wider and/or deeper range of the galvanic current passing through the skin. In one embodiment, the longest dimension of the galvanic particulates is at least twice (e.g., at least five times) the shortest dimension of such particulates.

The galvanic particulates may be of any shape, including but not limited to, spherical or non-spherical particulates or elongated or flattened shapes (e.g., cylindrical, fibers or flakes). In one embodiment, the average particle size of the galvanic particulates is from about 10 nanometers to about 500 micrometers, such as from about 100 nanometers to about 100 micrometers. As used herein, “average particle size” means the maximum dimension in at least one direction.

In one embodiment, the galvanic particulate comprises at least 90 percent by weight of conductive materials (e.g., the first conductive material and the second conductive material), such as at least 95 percent by weight, or at least 99 percent by weight, when a coating method is used for the production of the galvanic particulates.

Examples of combinations of first conductive materials/second conductive materials include (with a “/” sign representing an oxidized but essentially non-soluble form of the metal), but are not limited to, zinc-copper, zinc-copper/copper halide, zinc-copper/copper oxide, magnesium-copper, magnesium-copper/copper halide, zinc-silver, zinc-silver/silver oxide, zinc-silver/silver halide, zinc-silver/silver chloride, zinc-silver/silver bromide, zinc-silver/silver iodide, zinc-silver/silver fluoride, zinc-gold, zinc-carbon, magnesium-gold, magnesium-silver, magnesium-silver/silver oxide, magnesium-silver/silver halide, magnesium-silver/silver chloride, magnesium-silver/silver bromide, magnesium-silver/silver iodide, magnesium-silver/silver fluoride, magnesium-carbon, aluminum-copper, aluminum-gold, aluminum-silver, aluminum-silver/silver oxide, aluminum-silver/silver halide, aluminum-silver/silver chloride, aluminum-silver/silver bromide, aluminum-silver/silver iodide, aluminum-silver/silver fluoride, aluminum-carbon, copper-silver/silver halide, copper-silver/silver chloride, copper-silver/silver bromide, copper-silver/silver iodide, copper-silver/silver fluoride, iron-copper, iron-copper/copper oxide, copper-carbon iron-copper/copper halide, iron-silver, iron-silver/silver oxide, iron-silver/silver halide, iron-silver/silver chloride, iron-silver/silver bromide, iron-silver/silver iodide, iron-silver/silver fluoride, iron-gold, iron-conductive carbon, zinc-conductive carbon, copper-conductive carbon, magnesium-conductive carbon, and aluminum-carbon.

The first conductive material or second conductive material may also be an alloy, particularly the first conductive material. Non-limiting examples of alloys include alloys of zinc, iron, aluminum, magnesium, copper and manganese as the first conductive material and alloys of silver, copper, stainless steel and gold as second conductive material.

In one embodiment, the galvanic particulate comprises the first conductive material partially coated with several conductive materials, such as with a second and third conductive material. In a further embodiment, the particulate comprises at least 95 percent, by weight, of the first conductive material, the second conductive material, and the third conductive material. In one embodiment, the first conductive material is zinc, the second conductive material is copper, and the third conductive material is silver.

In one embodiment, the difference in the Standard Electrode Potentials (or simply, Standard Potential) of the first conductive material and the second conductive material is at least about 0.1 volts, such as at least 0.2 volts. In one embodiment, the materials that make up the galvanic couple have a Standard Potential difference equal to or less than about 3 volts. For example, for a galvanic couple comprised of metallic zinc and copper, the Standard Potential of zinc is −0.763V (Zn/Zn2+), and the Standard Potential of copper is +0.337 (Cu/Cu2+), the difference in the Standard Potential is therefore 1.100V for the zinc-copper galvanic couple. Similarly, for the magnesium-copper galvanic couple, Standard Potential of magnesium (Mg/Mg2+) is −2.363V, and the difference in the Standard Potential is therefore 2.700V. Additional examples of Standard Potential values of some materials suitable for use in galvanic particulates are: Ag/Ag+: +0.799V, Ag/AgCl/Cl−: 0.222V, and Pt/H2/H+: 0.000V. Pt may also be replaced by carbon or another conductive material. See, e.g., Physical Chemistry by Gordon M. Barrow, 4th Ed., McGraw-Hill Book Company, 1979, page 626.

In one embodiment, the first and second conductive electrodes are combined (e.g., the second conductive electrode is deposited to the first conductive electrode) by chemical, electrochemical, physical or mechanical process (such as electroless deposition, electric plating, vacuum vapor deposition, arc spray, sintering, compacting, pressing, extrusion, printing, and granulation) conductive metal ink (e.g., with polymeric binders), or other known metal coating or powder processing methods commonly used in powder metallurgy, electronics or medical device manufacturing processes, such as the methods described in the book Asm Handbook Volume 7: Powder Metal Technologies and Applications (Asm International Handbook Committee, edited by Peter W. Lee, 1998, pages 31-109, 311-320). In another embodiment, all the conductive electrodes are manufactured by chemical reduction processes (e.g., electroless deposition), sequentially or simultaneously, in the presence of reducing agent(s). Examples of reducing agents include phosphorous-containing reducing agents (e.g., a hypophosphite as described in U.S. Pat. Nos. 4,167,416 and 5,304,403), boron-containing reducing agents, and aldehyde- or ketone-containing reducing agents such as sodium tetrahydridoborate (NaBH₄) (e.g., as described in US 2005/0175649).

In one embodiment, the second conductive electrode is deposited or coated onto the first conductive electrode by physical deposition, such as spray coating, plasma coating, conductive ink coating, screen printing, dip coating, metals bonding, bombarding particulates under high pressure-high temperature, fluid bed processing, or vacuum deposition.

In one embodiment, the coating method is based on displacement chemical reaction, namely, contacting particles of the first conductive material (e.g., metallic zinc particles) with a solution containing a dissolved salt of the second conductive material (e.g. copper acetate, copper lactate, copper gluconate, or silver nitrate). In a further embodiment, the method includes flowing the solution over particles of the first conductive material (e.g., zinc powder) or through a packed powder of the first conductive material. In one embodiment, the salt solution is an aqueous solution. In another embodiment, the solution is contains an organic solvent, such as an alcohol, a glycol, glycerin or other commonly used solvent in pharmaceutical production to regulate the deposition rate of the second conductive material onto the surfaces of the first conductive material particles, therefore controlling the activity of the galvanic particulates produced.

In another embodiment, the galvanic particulates of the present invention may also be coated with other materials to protect the first and second conductive materials from degradation during storage (e.g., oxidation degradation from oxygen and moisture), or to modulate the electrochemical reactions and to control the electric current generated when in use. Exemplary coating materials include inorganic or organic polymers, natural or synthetic polymers, biodegradable or bioabsorbable polymers, silica, glass, various metal oxides (e.g., oxide of zinc, aluminum, magnesium, or titanium) and other inorganic salts of low solubility (e.g., zinc phosphate). Coating methods are known in the art of metallic powder processing and metal pigment productions, such as those described in U.S. Pat. No. 5,964,936; U.S. Pat. No. 5,993,526; U.S. Pat. No. 7,172,812; US 2006/0042509A1 and US 2007/0172438.

In one embodiment, the galvanic particulates are stored in anhydrous form, e.g., as a dry powder or as an essentially anhydrous non-conducting organic solvent composition (e.g., dissolved in polyethylene glycol, propylene glycol, glycerin, liquid silicone, and/or alcohol). In another embodiment, the galvanic particulates are embedded into an anhydrous carrier (e.g., inside a polymer). In yet another embodiment, the galvanic particulates are encapsulated in compositions of microcapsules, liposomes, or micelles, or embedded in the lipophilic phase of oil-in-water (O/W) or water-in-oil (W/O) types of emulsion systems (e.g., W/O lotion, W/O ointment, or O/W creams), as well as self-emulsifying compositions, in order to achieve self-life stability, retard the activation of the galvanic particulates, or prolong the action of galvanic particulates.

The skin care composition comprises at least one inorganic pigment. Inorganic pigments include iron oxides, including red and yellow iron oxides, titanium dioxide, ultramarine and chromium or chromium hydroxide colors, and mixtures thereof. Specifically excluded from the term “inorganic pigments” are particulates that consist of or consist essentially of fillers such as mica, talc, silica, or clays. Such fillers generally have relatively low opacity or hiding power compared to titanium dioxide. Also, specifically excluded from the term “inorganic pigments” are lake pigments and interference pigments described below. In one embodiment, the skin care composition comprises at least about 0.05 weight percent of inorganic pigments. In another embodiment, the skin care composition comprises no greater than about 4 weight percent of inorganic pigments. In another embodiment the skin care composition comprises about 0.05% to 4% weight percent inorganic pigments. In another embodiment, the skin care composition comprises no greater than about 3 weight percent of inorganic pigments. In another embodiment, the skin care composition comprises about 1 to about 3 weight percent of inorganic pigments.

The inorganic pigment comprises at least about 60 weight percent titanium dioxide. Preferably, the inorganic pigment comprises at least about 85 weight percent titanium dioxide.

The skin care composition also comprises at least one lake pigment. Examples of lake pigments include organic dyes such as azo, indigoid, triphenylmethane, anthraquinone, and xanthine dyes that are designated as D&C and FD&C blues, browns, greens, oranges, reds, yellows, etc., precipitated onto inert binders such as insoluble salts. In one embodiment, the lake pigment is selected from Red 6, Red 7, Yellow 5 and Blue #1. In another embodiment, the skin care composition comprises at least about 0.02 weight percent of lake pigments. In another embodiment, the skin care composition comprises no greater than about 1.5 weight percent of lake pigments. In a further embodiment, the skin care composition comprises about 0.02 to 1.5 weight percent of lake pigments. In another embodiment, the skin care composition comprises about 0.2 to about 1 weight percent of lake pigments.

The skin care composition further comprises at least one interference pigment.

Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF).

In one embodiment, the skin care composition comprises at least about 0.05 weight percent of an interference pigment. In another embodiment, the skin care composition comprises no greater than about 4.5 weight percent of an interference pigment. In another embodiment, the skin care composition comprises about 0.05 to 4.5 weight percent of an interference pigment. In another embodiment, the skin care composition comprises about 0.2 to about 4.25 weight percent of interference pigments.

These skin care composition may comprise any one of a variety of cosmetically-acceptable topical carriers including, but not limited to solutions, emulsions (e.g., microemulsions and nanoemulsions), gels, solids and liposomes. The following are non-limitative examples of such topical carriers. Other topical carriers can be formulated by those of ordinary skill in the art.

Solutions typically include an aqueous or organic solvent (e.g., from about 50% to about 99.99% or from about 90% to about 99% of a cosmetically acceptable aqueous or organic solvent). Examples of suitable organic solvents include propylene glycol, polyethylene glycol (200-600), polypropylene glycol (425-2025), glycerol, 1,2,4-butanetriol, sorbitol esters, 1,2,6-hexanetriol, ethanol, and mixtures thereof.

The solution may comprise an emollient, for example about 2% to about 50% by weight of an emollient(s). As used herein, “emollients” refer to materials used for the prevention or relief of dryness, such as by preventing the transepidermal loss of water from the skin. Examples of emollients include but are not limited to vegetable oils, mineral oils, fatty esters, and the like.

Lotions can be made from such solutions. Lotions typically contain from about 1% to about 20% (e.g., from about 5% to about 10%) of an emollient(s) and from about 50% to about 90% (e.g., from about 60% to about 80%) of water.

Creams typically contain from about 5% to about 50% (e.g., from about 10% to about 20%) of an emollient(s) and from about 45% to about 85% (e.g., from about 50% to about 75%) of water.

The skin care composition may be formulated as an emulsion, for example containing from about 1% to about 10% by weight (e.g., from about 2% to about 5% by weight) of an emulsifier(s). Emulsifiers may be nonionic, anionic or cationic. Examples of suitable emulsifiers include those typically identified as such in the art of personal care and cosmetic formulations.

Lotions and creams can be formulated as emulsions. Typically such lotions contain from 0.5% to about 5% of an emulsifier(s). Such creams typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion compositions, such as lotions and creams, of the oil-in-water type and water-in-oil type are well-known in the cosmetic art and are useful. Multiphase emulsion compositions, such as the water-in-oil-in-water type or the oil-in-water-in-oil type, are also useful. In general, such single or multiphase emulsions contain water, emollients, and emulsifiers as essential ingredients.

The composition can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

In one embodiment, the composition comprises an additional active agent. As used herein, “additional active agent” means a compound (e.g., synthetic or natural) that provides a cosmetic or therapeutic effect on the skin, such as a therapeutic drug or cosmetic agent. Examples of therapeutic drugs include small molecules, peptides, proteins, nucleic acid materials, and nutrients such as minerals and extracts. The amount of the additional active agent in the composition will depend on the active agent, other ingredients present in the composition, and the desired benefits of the composition. In one embodiment, the composition contains a safe and effective amount of the additional active agent, for example, from about 0.001 percent to about 20 percent, by weight, such as from about 0.01 percent to about 10 percent, by weight, of the composition.

The galvanic particulates can be combined with an additional active agent (such as antimicrobial agents, anti-inflammatory agents, and analgesic agents) to enhance or potentiate the biological or therapeutic effects of that active agent. In another embodiment, the galvanic particulates can also be combined with other substances to enhance or potentiate the activity of the galvanic particulates. Substances that can enhance or potentiate the activity of the galvanic particulates include, but are not limited to, organic solvents (such as alcohols, glycols, glycerin, polyethylene glycols and polypropylene glycol), surface active agents (such as nonionic surfactants, zwitterionic surfactants, anionic surfactants, cationic surfactants and polymeric surfactants), and water-soluble polymers. For example, the galvanic particulates can form conjugates or composites with synthetic or natural polymers including by not limited to proteins, polysaccharides, hyaluronic acid of various molecular weight, hyaluronic acid analogs, polypeptides, and polyethylene glycols.

In one embodiment, the composition contains a chelator or chelating agent. Examples of chelators include, but are not limited to, amino acids such as glycine, lactoferrin, edetate, citrate, pentetate, tromethamine, sorbate, ascorbate, deferoxamine, derivatives thereof, and mixtures thereof. Other examples of chelators useful are disclosed in U.S. Pat. No. 5,487,884 and PCT Publication Nos. 91/16035 and 91/16034.

In one embodiment, the composition contains an anti-aging agent. Examples of suitable anti-aging agents include, but are not limited to: inorganic sunscreens such as titanium dioxide and zinc oxide; organic sunscreens such as octyl-methoxy cinnamates; retinoids; dimethylaminoathanol (DMAE); alpha hydroxy acids and their precursors such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, isopropyl pyruvate, methyl pyruvate, mucic acid, pyruvic acid, saccharic acid, saccaric acid 1,4-lactone, tartaric acid, and tartronic acid; beta hydroxy acids such as beta-hydroxybutyric acid, beta-phenyl-lactic acid, and beta-phenylpyruvic acid; tetrahydroxypropyl ethylene-diamine, N,N,N′,N′-Tetrakis(2-hydroxypropyl)ethylenediamine (THPED); and botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, and safflower; and salts, derivatives and prodrugs thereof.

In one embodiment, the composition contains a buffering agent such as citrate buffer, phosphate buffer, lactate buffer, gluconate buffer, or gelling agents, thickeners, or polymers.

In one embodiment, the composition contains a fragrance.

In another embodiment, the composition comprises an anti-inflammatory agent. Examples of anti-inflammatory agents, include, but are not limited to, suitable steroidal anti-inflammatory agents such as corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and salts are prodrugs thereof. In one embodiment, the steroidal anti-inflammatory agent is hydrocortisone. Non-steroidal anti-inflammatory agents may also be used.

Other optional ingredients include abrasives, absorbents, aesthetic components such as skin sensates, astringents, anti-acne agents, anti-caking agents, antifoaming agents, antimicrobial agents, antioxidants, binders, biological additives, buffering agents, bulking agents, chemical additives, cosmetic biocides, denaturants, drug astringents, external analgesics, enzymes, emulsifiers, film formers or materials, e.g., polymers, for aiding the film-forming properties and substantivity of the composition, opacifying agents, other pigments, pH adjusters, propellants, reducing agents, sequestrants, skin bleaching and lightening agents, skin-conditioning agents (e.g., humectants, including miscellaneous and occlusive), skin soothing and/or healing agents, skin treating agents, structuring agents, and thickeners.

The skin care composition may contain water or may alternatively be anhydrous, i.e., containing organic and/or silicone solvents, oils, lipids and waxes. In one embodiment, the skin care composition is anhydrous. In another embodiment, the skin care composition is an anhydrous primer.

In another embodiment, the skin care composition contains one or more crosslinked organopolysiloxane gels. Suitable organopolysiloxane polymer gels include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200, and hybrid silicone powders that contain a phenyl group such as Shin-Etsu's KSP-300; and Dow Corning's DC 9506.

Preferred organopolysiloxane gels include dimethicone/vinyl dimethicone crosspolymers, including those commercially available from Dow Corning (DC 9040 and DC 9041), General Electric (SFE 839), Shin Etsu (KSG-15, 16, 18 [dimethicone/phenyl vinyl dimethicone crosspolymer] and KSG-21 [dimethicone copolyol crosspolymer]), Grant Industries (GRANSIL line of materials), lauryl dimethicone/vinyl dimethicone crosspolymers supplied by Shin Etsu (e.g., KSG-41, KSG-42, KSG-43, and KSG-44), and lauryl dimethicone/dimethicone copolyol crosspolymers also supplied by Shin-Etsu (e.g., KSG-31, KSG-32, KSG-33, and KSG-34). Additional suitable polymers from Shin-Etsu include KSG-210, -310, 320, 330, and 340. Crosslinked organopolysiloxane polymer gel networks useful in the present invention and processes for making them are further described in U.S. Pat. No. 4,970,252, U.S. Pat. No. 5,760,116, U.S. Pat. No. 5,654,362 and Japanese Patent Application JP 61-18708.

Water and oil dispersible clays may be useful to thicken water or oil phases of the skin care composition. Water dispersible clays comprise for example bentonite and hectorite, such as BENTONE EW, LT from Rheox; magnesium aluminum silicate, such as VEEGUM from Vanderbilt Co., attapulgite such as ATTASORB or PHARMASORB from Engelhard, Inc.; laponite and montmorrilonite, such as GELWHITE from ECC America, and mixtures thereof. Oil dispersible clays include quaternium-18 bentonite, such as BENTONE 34 and 38 from Rheox; the CLAYTONE Series from ECC America; quaternium-18 hectorite, such as BENTONE gels from Rheox; and mixtures thereof. Other particulate or organic thickeners may also be used provided they do not compromise the function or aesthetics of the color cosmetic composition.

Film forming agents may be optionally included in the compositions of the present invention to aid film substantivity and adhesion to the skin. Improving the wear and non-transfer performance of the present compositions is quite desirable. Water-soluble, water insoluble, and water dispersible film forming agents can be used.

The compositions of the present invention can be generally prepared by conventional methods known in the cosmetic art. Such methods typically involve mixing of the ingredients in one or more steps to a relatively uniform state, with or without heating, cooling, application of vacuum, and the like.

The following non-limiting examples further illustrate the invention.

### EXAMPLES

Cosmetic primers according to the invention were made with the ingredients shown in the Table below. The primers contained galvanic particulates, but had pleasant, consumer acceptable shades.

| INGREDIENT | COMPOSITION 1 | COMPOSITION 2 | COMPOSITION 3 | COMPOSITION 4 |
|---|---|---|---|---|
| Phase A | | | | |
| Dimethicone | 20 | 25 | 25 | 30 |
| crosspolymer | | | | |
| TiO₂ | 1.6 | 2.5 | 2.8 | 2.9 |
| Red Iron Oxide | 0.2 | 0.04 | 0 | 0 |
| FD&C Yellow | 0.2 | 0.34 | 0.6 | 0.7 |
| No. 5 | | | | |
| Aluminum | | | | |
| Lake (And) | | | | |
| Isopropyl | | | | |
| Titanium | | | | |
| Triisostearate | | | | |
| Red 6 Lake | 0.07 | 0 | 0 | 0.05 |
| (And) | | | | |
| Isopropyl | | | | |
| Titanium | | | | |
| Triisostearate | | | | |
| Talc | 4 | 4 | 4 | 4 |
| Lauryl PEG-9 | 1 | 1 | 1 | 1 |
| Polydimethyl- | | | | |
| siloxyethyl | | | | |
| dimethicone) | | | | |
| Phase B | | | | |
| Dimethicone | 33 | 30 | 29.7 | 20.2 |
| crosspolymer | | | | |
| Dimethicone/ | 6 | 8 | 7 | 12 |
| Vinyl | | | | |
| Dimethicone | | | | |
| Crosspolymer, | | | | |
| Methyl | | | | |
| Trimethicone | | | | |
| TMF 1.5 | 12 | 11.2 | 11 | 8 |
| Isododecane | 8 | 9.8 | 9 | 10 |
| Phase C | | | | |
| C30-45 Alkyl | 0.53 | 0.52 | 0.7 | 0.6 |
| Methicone and | | | | |
| C30-45 Olefin | | | | |
| Phase D | | | | |
| Dimethicone/ | 2 | 0.3 | 1 | 1 |
| Vinyl | | | | |
| Dimethicone | | | | |
| Crosspolymer | | | | |
| (and) Silica | | | | |
| Silica | 0.4 | 0.4 | 0.4 | 0.4 |
| Nylon | 2 | 2 | 2 | 2 |
| Silica | 1 | 1 | 1 | 1 |
| Hybrid silica | 1 | 1 | 1 | 1 |
| Silica | 0.9 | 0.7 | 0.7 | 0.7 |
| Galvanic | 2 | 2 | 2 | 2 |
| Particulates | | | | |
| Mica (And) | 3 | 0 | 0.5 | 1 |
| Titanium | | | | |
| Dioxide | | | | |
| Mica (And) | 1 | | 0.5 | 1 |
| Titanium | | | | |
| Dioxide | | | | |
| Mica (and) | 0.1 | 0.2 | 0.1 | 0.2 |
| Bismuth | | | | |
| Oxychloride | | | | |
| (and) Iron | | | | |
| Oxides | | | | |
| Mica (and) | | | | 0.2 |
| Bismuth | | | | |
| Oxychloride | | | | |
| (and) Carmine | | | | |
| | 100 | 100 | 100 | 99.95 |

Each primer was made as follows. First, Phase A was ground using a roller mill. Next, Phase A was added to Phase B. The mixture was heated to 60° C. and mixed until homogenous. Phase C was then added, and the resulting mixture was heated to 75° C. until the wax melted. The batch was then covered to ensure there was no solvent loss. Finally, Phase D was added at 65-70° C. and the ingredients were mixed until homogenous.

We claim:

1. A skin care composition comprising:
a) an active ingredient having an undesirable color;
b) about 0.05% to 4 weight percent of at least one inorganic pigment, wherein said inorganic pigment comprises at least 60 weight percent titanium dioxide;
c) about 0.02 to 1.5 weight percent of at least one lake pigment; and
d) about 0.05 to 4.5 weight percent of at least one interference pigment.

2. The skin care composition of claim 1, wherein said active ingredient has a metallic, grey, or dark color.

3. The skin care composition of claim 1, wherein said active ingredient comprises galvanic powder.

4. The skin care composition of claim 1, wherein said active ingredient comprises a plant extract.

5. The skin care composition of claim 1, wherein said active ingredient comprises feverfew extract.

6. The skin care composition of claim 1, wherein said skin care composition is a color cosmetic.

7. The skin care composition of claim 1, wherein said skin care composition is a primer.
